(19) United States
(12) Patent Application Publication
(10) Pub. No.: US 2024/0277662 A1
(43) Pub. Date: Aug. 22, 2024

(54) Administration of a Compound to Individuals with Hepatic Impairment

(71) Applicant: Arena Pharmaceuticals, Inc., New York, NY (US)

(72) Inventor: Caroline A. Lee, Carlsbad, CA (US)

(73) Assignee: Arena Pharmaceuticals, Inc., New York, NY (US)

(21) Appl. No.: 18/566,862

(22) PCT Filed: Jun. 14, 2022
(86) PCT No.: PCT/US2022/033471

§ 371 Date: Dec. 4, 2023

Related U.S. Application Data
(60) Provisional application No. 63/218,032, filed on Jul. 2, 2021.

(51) Int. Cl. A61K 31/404

(52) U.S. Cl. CPC A61K 31/404

(57) ABSTRACT

Provided are methods useful in the prophylaxis or treatment of an S1P1 modulated disease in different populations of individuals, including those with hepatic impairment. In some methods, etrasimod is prescribed or administered to an individual in need of treatment wherein the dose is not adjusted for the patient having mild, moderate, or severe hepatic impairment.

This application claims the benefit of U.S. Provisional Application No. 63/218,032, filed Jul. 2, 2021, which is incorporated herein by reference in its entirety.

Provided herein are methods useful in the prophylaxis or treatment of an S1P1 modulated disease in different populations of individuals, including those with hepatic impairment. In some methods provided herein, etrasimod is prescribed or administered to an individual in need of treatment if they do not have severe hepatic impairment.

Etrasimod, an oral, selective sphingosine 1-phosphate receptor 1, 4, 5 modulator, is in development for ulcerative colitis, atopic dermatitis, alopecia areatea, eosinophilic esophagitis, and Crohn's disease.

Hepatic impairment is a condition wherein normal functioning of the liver is reduced. Hepatic impairment can be acute, with rapid onset, or chronic. Chronic hepatic impairment, or cirrhosis, can occur from many causes, such as excessive consumption of alcohol, hepatitis, autoimmune disease, heredity, or metabolism, or can be idiopathic. Liver damage is generally irreversible, and treatment consists of prevention of progression and treatment of symptoms. In severe cases, liver transplant is the only option. Hepatic impairment can exhibit no significant symptoms, or may be characterized by such symptoms as reduced ability for the blood to clot (coagulopathy) and brain dysfunction (encephalopathy), fluid retention in the abdominal cavity, increased infection risk, hypogonadism, change in liver size, jaundice, and increased sensitivity to medication.

Liver disease can alter drug disposition and pharmacokinetics, reducing hepatic or biliary clearance. Plasma protection binding may also be affected, which could potentially influence the efficacy and safety of a drug.

Applicants have disclosed herein the interaction of hepatic impairment with the pharmacokinetics, tolerability and safety of etrasimod in a formal pharmacokinetic study in subjects with hepatic impairment.

There exists a need for safely treating individuals who are in need of treatment with etrasimod including individuals with hepatic impairment. The present disclosure satisfies this need and provides related advantages as well.

### SUMMARY

Provided is a method of treating an S1P1 receptor-associated disorder in a patient having hepatic impairment, comprising: administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt thereof.

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” means (R)-2-(7-(4-cyclopentyl-3-(trifluoromethyl)benzyloxy)-1,2,3,4-tetrahydrocyclopenta[b]indol-3-yl)acetic acid including crystalline forms thereof.

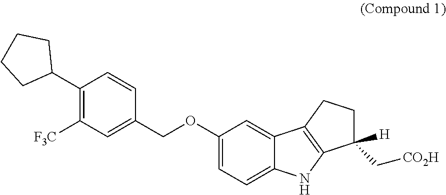 [SMILES: O=C(O)C[C@H]1CCc2c1[nH]c1ccc(OCc3ccc(C4CCCC4)c(C(F)(F)F)c3)cc21]

See PCT patent application, Serial No. PCT/US2009/004265 hereby incorporated by reference in its entirety. As a non-limiting example, Compound 1 may be present as an anhydrous, non-solvated crystalline form as described in WO 2010/011316 (incorporated by reference herein in its entirety). As another non-limiting example, an L-arginine salt of Compound 1 may be present as an anhydrous, non-solvated crystalline form as described in WO 2010/011316 and WO 2011/094008 (each of which is incorporated by reference herein in its entirety). As another non-limiting example, a calcium salt of Compound 1 may be present as a crystalline form as described in WO 2010/011316 (incorporated by reference herein in its entirety). Compound 1 is referred to in literature as etrasimod or APD334.

Compound 1, or a pharmaceutically acceptable salt thereof, is an orally administered, selective, synthetic sphingosine 1-phosphate (SIP) receptor 1, 4, 5 modulator. To date, Compound 1, or a pharmaceutically acceptable salt thereof, has been found to be safe and well-tolerated in approximately 281 adult subjects treated at various doses. Its safety and tolerability have been evaluated in Phase 1 studies with healthy adult subjects at single doses up to 5 mg and repeated doses up to 4 mg once daily (QD). In a Phase 2 dose-ranging study in UC patients, treatment with 2 mg QD for 12 weeks led to clinically meaningful and statistically significant endoscopic and symptomatic improvements versus placebo. Sustained beneficial effects of were observed for up to 46 weeks in the subsequent open-label extension study. Methods of use of Compound 1 are disclosed in US Patent Application Publication No. 2018-0263958, PCT Publication No. WO2021/102357, PCT Publication No. WO2021/067506, and PCT Patent Application No. PCT/US2021/012367, which are hereby incorporated by reference in their entirety.

ADMINISTERING: As used herein, “administering” means to provide a compound or other therapy, remedy, or treatment such that an individual internalizes a compound.

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to an individual. In this case the health care practitioner may or may not provide a prescription for the compound, dosage regimen, or treatment. Further, the health care practitioner may or may not provide the recommended compound or treatment. For example, the health care practitioner can advise the individual where to obtain the compound without providing the compound. In some embodiments, a health care practitioner can provide a prescription for the compound, dosage regimen, or treatment to the individual. For example, a health care practitioner can give a written or oral prescription to an individual. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the internet to a pharmacy or a dispensary. In some embodiments, a sample of the compound or treatment can be given to the individual. As used herein, giving a sample of a compound constitutes an implicit prescription for the compound. Different health care systems around the world use different methods for prescribing and/or administering compounds or treatments and these methods are encompassed by the disclosure.

A prescription can include, for example, an individual's name and/or identifying information such as date of birth. In addition, for example, a prescription can include: the medication name, medication strength, dose, frequency of administration, route of administration, number or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a condition described herein. In addition, a healthcare practitioner can include anyone who can recommend, prescribe, administer, or prevent an individual from receiving a compound or drug including, for example, an insurance provider.

PREVENT, PREVENTING, OR PREVENTION: As used herein, the term “prevent,” “preventing”, or “prevention,” such as prevention of a particular disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to an individual who may ultimately manifest at least one symptom of a disease or condition but who has not yet done so. Such individuals can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein, the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting, even if the severity of each is less than when experienced alone.

INTOLERANCE: As used herein, “intolerance” means significant toxicities and/or tolerability issues that led to a reduction in dose or discontinuation of the medication. “Intolerance” can be replaced herein with the term “unable to tolerate.”

ADVERSE EVENT: As used herein, an “adverse event” is an untoward medical occurrence that is associated with treatment with Compound 1 or a pharmaceutically acceptable salt thereof. In one embodiment, an adverse event is selected from: leukopenia, constipation, diarrhea, nausea, abdominal pain, neutropenia, vomiting, back pain, and menstrual disorder. In one embodiment, an adverse event is heart block, for example, a first-degree atrioventricular heart block. In one embodiment, an adverse event is an acute heart rate reduction. In one embodiment, an adverse event is an abnormal pulmonary function test finding, such as an FEV1 below 80%, FVC. In one embodiment, an adverse event is macular edema.

IN NEED OF TREATMENT and IN NEED THEREOF: As used herein, “in need of treatment” and “in need thereof” when referring to treatment are used interchangeably to mean a judgment made by a caregiver (e.g. physician, nurse, nurse practitioner, etc.) that an individual requires or will benefit from treatment. This judgment is made based on a variety of factors that are in the realm of a caregiver's expertise, but that includes the knowledge that the individual is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

INDIVIDUAL: As used herein, “individual” means any human. In some embodiments, a human individual is referred to a “subject” or “patient.”

ACUTE HEART RATE REDUCTION: As used herein, “acute heart rate reduction” means a heart rate decrease from normal sinus rhythm of, for example, 10 or more beats per minute (bpm), such as less than about 5 bpm, e.g., less than about 4 bpm or less than about 3 bpm or less than 2 bpm, that is maximal within a few hours, for example 1-3 hours, after drug administration, and thereafter the heart rate returns towards the pre-dose value.

NORMAL SINUS RHYTHM: As used herein, “normal sinus rhythm” means the sinus rhythm of the individual when not undergoing treatment. The evaluation of normal sinus rhythm is within the ability of a physician. A normal sinus rhythm will generally give rise to a heart rate in the range from 60-100 bpm.

DOSE: As used herein, “dose” means a quantity of Compound 1, or a pharmaceutically acceptable salt thereof, given to the individual for treating or preventing the disease or disorder at one specific time.

THERAPEUTICALLY EFFECTIVE AMOUNT: As used herein, “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician. In some embodiments, the therapeutically effective amount is the standard dose.

PHARMACEUTICAL COMPOSITION: As used herein, “pharmaceutical composition” means a composition comprising at least one active ingredient, such as Compound 1, including but not limited to, salts of Compound 1, whereby the composition is amenable to investigation for a specified, efficacious outcome. Those of ordinary skill in the art will understand and appreciate the techniques appropriate for determining whether an active ingredient has a desired efficacious outcome based upon the needs of the artisan.

HEPATIC IMPAIRMENT: Hepatic impairment can be measured by several methods. For example, hepatic impairment can be measured based on Child-Pugh (CP) score. Mild hepatic impairment is defined as a CP score from 5-6. Moderate hepatic impairment is defined as a CP score from 7-9. Severe hepatic impairment is defined as a CP score of 10-14. The Child-Pugh score is known to those of skill in the art, and is a score based on five clinical measures of hepatic impairment, including levels of total bilirubin, serum albumin, PT INR, ascites, and hepatic encephalopathy. Each measure is given a ranking of 1, 2, or 3, and the sum of the five rankings is the Child-Pugh Score.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed by Berge et al., Journal of Pharmaceutical Sciences, 66:1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

It is understood that when the phrase “pharmaceutically acceptable salts, solvates and hydrates” or the phrase “pharmaceutically acceptable salt, solvate, or hydrate” is used when referring to Compound 1, it embraces pharmaceutically acceptable solvates and/or hydrates of Compound 1, pharmaceutically acceptable salts of Compound 1, as well as pharmaceutically acceptable solvates and/or hydrates of pharmaceutically acceptable salts of Compound 1. It is also understood that when the phrase “pharmaceutically acceptable solvates and hydrates” or the phrase “pharmaceutically acceptable solvate or hydrate” is used when referring to Compound 1 that are salts, it embraces pharmaceutically acceptable solvates and/or hydrates of such salts. It will be apparent to those skilled in the art that the dosage forms described herein may comprise, as the active component, either Compound 1 or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, DE), Avantium Technologies (Amsterdam) and Aptuit (Greenwich, CT).

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e., one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all the steps, features, compositions and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination. For example, a method that recites prescribing and/or administering Compound 1 or a pharmaceutically acceptable salt thereof can be separated into two methods; one method reciting prescribing Compound 1 or a pharmaceutically acceptable salt thereof and the other method reciting administering Compound 1 or a pharmaceutically acceptable salt thereof. In addition, for example, a method that recites prescribing Compound 1 or a pharmaceutically acceptable salt thereof and a separate method of the invention reciting administering Compound 1 or a pharmaceutically acceptable salt thereof can be combined into a single method reciting prescribing and/or administering Compound 1 or a pharmaceutically acceptable salt thereof.

In some embodiments, the individual is administered an amount equivalent to about 0.5 to about 5.0 mg of Compound 1. In some embodiments, the individual is administered an amount equivalent to 2 mg of Compound 1. In some embodiments, the individual is administered an amount equivalent to 2.25 mg of Compound 1. In some embodiments, the individual is administered an amount equivalent to 2.5 mg of Compound 1. In some embodiments, the individual is administered an amount equivalent to 2.75 mg of Compound 1. In some embodiments, the individual is administered an amount equivalent to 3 mg of Compound 1. In some embodiments, the individual is administered an amount equivalent to 1 mg of Compound 1.

In some embodiments, the individual is administered Compound 1 or a pharmaceutically acceptable salt thereof at least one month, such as one month, two months, three months, four months, etc. In some embodiments, the individual is administered Compound 1 or a pharmaceutically acceptable salt thereof for least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the second time period is indefinite, e.g., chronic administration.

In some embodiments, the individual is administered an amount equivalent to 2 mg of Compound 1 for a first time period and subsequently an amount equivalent to 3 mg of Compound 1 for a second time period. In some embodiments, the first time period is at least one month, such as one month, two months, three months, four months, etc. In some embodiments, the first time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the second time period is at least one month, such as one month, two months, three months, four months, etc. In some embodiments, the second time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the second time period is indefinite, e.g., chronic administration. In some embodiments, the dosage for the first time period is not adjusted during the first time period. In some embodiments, the dosage for the second time period is not adjusted during the second time period.

In some embodiments, the individual is administered an amount equivalent to 3 mg of Compound 1 for a first time period and subsequently an amount equivalent to 2 mg of Compound 1 for a second time period. In some embodiments, the first time period is at least one month, such as one month, two months, three months, four months, etc. In some embodiments, the first time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the second time period is at least one month, such as one month, two months, three months, four months, etc. In some embodiments, the second time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the second time period is indefinite, e.g., chronic administration. In some embodiments, the dosage for the first time period is not adjusted during the first time period. In some embodiments, the dosage for the second time period is not adjusted during the second time period.

In some embodiments, the standard dose is administered without titration. In some embodiments, the standard dose is administered without titration; and the individual does not experience a severe related adverse event. In some embodiments, the standard dose is administered without requiring titration to avoid first-dose effect seen with other SIP receptor modulators.

In some embodiments, the dosage form is administered under fasted conditions. In some embodiments, the dosage form is administered under fed conditions.

In some embodiments, the method is non-gender specific.

In some embodiments, Compound 1, or a pharmaceutically acceptable salt thereof, is administered in combination with a second therapeutic agent or therapy, wherein the second therapeutic agent or therapy is selected from an IL-1beta inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a glucocorticoid, a proton pump inhibitor, a NSAID, allergen removal and diet management.

In some embodiments, Compound 1, or a pharmaceutically acceptable salt thereof, was previously administered at least one therapeutic agent or therapy, wherein the therapeutic agent or therapy is selected from an IL-1beta inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a glucocorticoid, a proton pump inhibitor, a NSAID, allergen removal and diet management.

In some embodiments, the individual is, or was, treated with an IL-1beta inhibitor. In some embodiments, the IL-1beta inhibitor is anakinra, rilonacept, or canakinumab.

In some embodiments, the individual is, or was, treated with an IL-5 inhibitor. In some embodiments, the IL-5 inhibitor is benralizumab, mepolizumab or reslizumab.

In some embodiments, the individual is treated with an IL-9 inhibitor.

In some embodiments, the individual is, or was, treated with an IL-13 inhibitor. In some embodiments, the IL-13 inhibitor is lebrikizumab, RPC4046, or tralokinumab.

In some embodiments, the individual is, or was, treated with an IL-17 inhibitor. In some embodiments, the IL-17 inhibitor is ixekizumab or brodalumab.

In some embodiments, the individual is, or was, treated with an IL-25 inhibitor.

In some embodiments, the individual is, or was, treated with a TNFα inhibitor. In some embodiments, the TNFα inhibitor is SIMPONI® (golimumab), REMICADE® (infliximab), HUMIRA® (adalimumab), or CIMZIA® (certolizumab pegol).

In some embodiments, the individual is, or was, treated with an eotaxin-3 inhibitor. In some embodiments, the individual is, or was, treated with an IgE inhibitor. In some embodiments, the IgE inhibitor is omalizumab.

In some embodiments, the individual is, or was, treated with a prostaglandin D2 inhibitor. In some embodiments, the individual is, or was, treated with an immunosuppressant. In some embodiments, the immunosuppressant is AZASAN® (azathioprine), IMURAN® (azathioprine), GENGRAF® (cyclosporine), NEORAL® (cyclosporine), or SANDIMMUNE® (cyclosporine). Immunosuppressants also may be referred to as immunosuppressives or immunosuppressive agents.

In some embodiments, the individual is, or was, treated with a proton pump inhibitor. In some embodiments, the proton pump inhibitor is omeprazole, pantoprazole, esomeprazole, or dexlansoprazole.

In some embodiments, the individual is, or was, treated with a glucocorticoid. In some embodiments, the glucocorticoid is UCERIS® (budesonide); DELTASONE® (prednisone), MEDROL® (methylprednisolone), or hydrocortisone. Glucocorticosteroids also may be referred to as glucocorticoid or corticosteroids.

In some embodiments, the individual is, or was, treated with a NSAID. In some embodiments, the NSAID is aspirin, celecoxib, diclofenac, diflunisal, etodolac, ibuprofen, indomethacin, ketoprofen, ketorolac, nabumetone, naproxen, oxaprozin, piroxicam, salsalate, sulindac, or tolmetin.

In some embodiments, the individual has had an inadequate response with, lost response to, or been intolerant to a conventional therapy. In some embodiments, the individual has had an inadequate response to conventional therapy. In some embodiments, the individual has lost response to conventional therapy. In some embodiments, the individual has been intolerant to conventional therapy. In some embodiments, the conventional therapy is selected from: an IL-1beta inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a glucocorticoid, a proton pump inhibitor, a NSAID, allergen removal and diet management. In some embodiments, the prior conventional therapy is referred to as prior treatment.

In some embodiments, the individual had an inadequate response with, lost response to, or was intolerant to the at least one therapeutic agent or therapy. In some embodiments, the individual has demonstrated an inadequate response to, loss of response to, or intolerance of to the at least one therapeutic agent or therapy. In some embodiments, the individual had demonstrated, over the previous 3-month period, an inadequate response to, loss of response to, or intolerance of the at least one therapeutic agent or therapy. In some embodiments, the individual had demonstrated, over the previous 6-month period, an inadequate response to, loss of response to, or intolerance of the at least one therapeutic agent or therapy. In some embodiments, the individual had demonstrated, over the previous 9-month period, an inadequate response to, loss of response to, or intolerance of the at least one therapeutic agent or therapy. In some embodiments, the individual had demonstrated, over the previous 1-year period, an inadequate response to, loss of response to, or intolerance of the at least one therapeutic agent or therapy. In some embodiments, the individual had demonstrated, over the previous 2-year period, an inadequate response to, loss of response to, or intolerance of the at least one therapeutic agent or therapy. In some embodiments, the individual had demonstrated, over the previous 3-year period, an inadequate response to, loss of response to, or intolerance of the at least one therapeutic agent or therapy. In some embodiments, the individual had demonstrated, over the previous 4-year period, an inadequate response to, loss of response to, or intolerance of the at least one therapeutic agent or therapy. In some embodiments, the individual had demonstrated, over the previous 5-year period, an inadequate response to, loss of response to, or intolerance of the at least one therapeutic agent or therapy.

In some embodiments, prior to the treatment, the individual will have been administered proton pump inhibitor therapy. In some embodiments, prior to the treatment, the individual had an inadequate response with, lost response to, or was intolerant to proton pump inhibitor therapy. In some embodiments, the individual will have been on a stable dose of proton pump inhibitor therapy for at least two months.

In some embodiments, prior to the treatment, the individual will not have severe strictures.

In some embodiments, the method further comprises monitoring for adverse events during the administration of Compound 1, or a pharmaceutically acceptable salt thereof, and optionally, interrupting or terminating the administration of Compound 1, or a pharmaceutically acceptable salt thereof.

In some embodiments, the treatment further comprises monitoring heart rate during the administration, monitoring pulmonary function during the administration, or monitoring liver function during the administration.

In some embodiments, the treatment further comprises monitoring heart rate during the administration.

In some embodiments, the treatment further comprises monitoring pulmonary function during the administration.

In some embodiments, the treatment further comprises monitoring liver function during the administration.

In some embodiments, the method reduces the incidence and severity of adverse events resulting from the treatment of a condition described herein.

In some embodiments, the adverse event is a serious adverse event.

In some embodiments, the serious adverse event is selected from leukopenia, constipation, diarrhea, nausea, abdominal pain, neutropenia, vomiting, back pain, and menstrual disorder.

In some embodiments, the method results in no serious adverse events.

In some embodiments, the standard dose is administered without substantially inducing an acute heart rate reduction or heart block in the individual.

In some embodiments, Compound 1, or a pharmaceutically acceptable salt thereof, is administered without causing a reduction of more than 6 bpm in heart rate.

In some embodiments, Compound 1, or a pharmaceutically acceptable salt thereof, is administered without a first-dose effect on heart rate as seen with other SIP receptor modulators. In some embodiments, Compound 1, or a pharmaceutically acceptable salt thereof, is administered without a first-dose effect on AV conduction as seen with other SIP receptor modulators.

In some embodiments, a method of treating a S1P1 receptor-associated disorder in an individual comprises administering a therapeutically effective amount of Compound 1 or a pharmaceutically acceptable salt thereof to said individual, wherein said individual has previously been determined to have a level of hepatic impairment selected from the group consisting of: no hepatic impairment, mild hepatic impairment, moderate hepatic impairment, and severe hepatic impairment. In some embodiments, the individual has been diagnosed with mild hepatic impairment. In some embodiments, the individual has been diagnosed with moderate hepatic impairment. In some embodiments, the individual has been diagnosed with severe hepatic impairment.

In some embodiments, a method of treating a S1P1 receptor-associated disorder in an individual comprises administering a therapeutically effective amount of Compound 1 or a pharmaceutically acceptable salt thereof to said individual, wherein said individual has previously been determined to have a level of hepatic impairment selected from the group consisting of: no hepatic impairment, mild hepatic impairment, moderate hepatic impairment, and severe hepatic impairment. In some embodiments, the individual has been diagnosed with mild hepatic impairment. In some embodiments, the individual has been diagnosed with moderate hepatic impairment. In some embodiments, the individual has been diagnosed with severe hepatic impairment. In some embodiments, the S1P1 receptor-associated disorder is selected from the group consisting of ulcerative colitis, atopic dermatitis, alopecia areatea, eosinophilic esophagitis, and Crohn's disease. According to certain embodiments, a patient receives a starting dose of Compound 1, or a pharmaceutical salt thereof, but the dose is not adjusted. According to certain embodiments, no adjustment of dose is needed for the individual with hepatic impairment as compared to a patient having no hepatic impairment.

In some embodiments, a method of treating a S1P1 receptor-associated disorder in an individual comprises administering a therapeutically effective amount of Compound 1 or a pharmaceutically acceptable salt thereof to said individual, wherein said individual has previously been determined to have a Child-Pugh score in the range of 5-14. In some embodiments, the individual has been determined to have a Child-Pugh score of 5-6. In some embodiments, the individual has been determined to have a Child-Pugh score of 7-9. In some embodiments, the individual has been determined to have a Child-Pugh score of 10-14. In some embodiments, the individual has been determined to have a Child-Pugh score of 14 or higher. In some embodiments, the S1P1 receptor-associated disorder is selected from the group consisting of ulcerative colitis, atopic dermatitis, alopecia areatea, eosinophilic esophagitis, and Crohn's disease. According to certain embodiments, a patient receives a starting dose of Compound 1, or a pharmaceutical salt thereof, but the dose is not adjusted. According to certain embodiments, no adjustment of dose is needed for the individual with hepatic impairment as compared to a patient having no hepatic impairment.

### Example

A study evaluated the effect of hepatic impairment on the PK, safety, and tolerability of etrasimod.

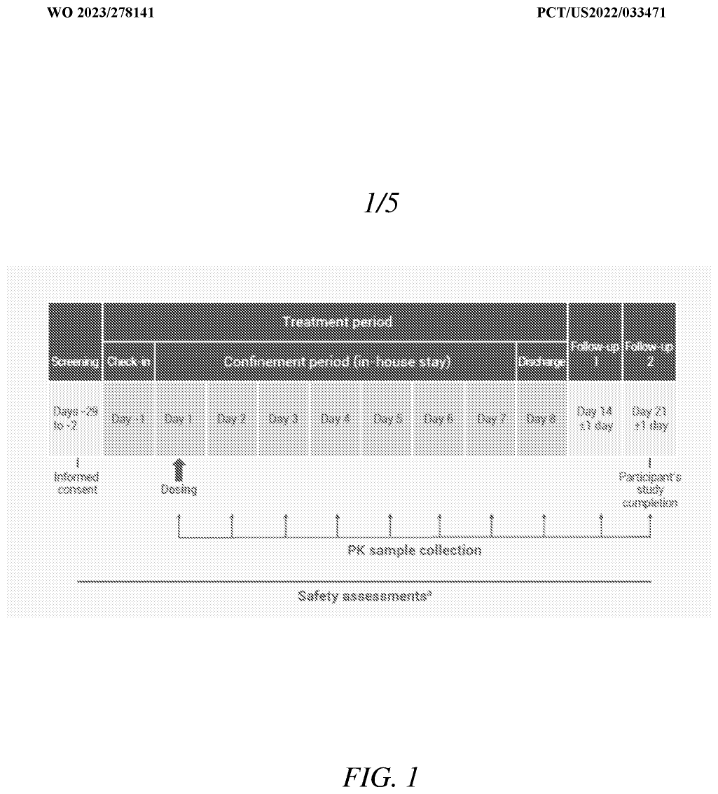

An open-label, parallel-group study included 36 patients (aged 18-80 years; body mass index ≥18) with hepatic impairment, based on Child-Pugh score at screening (mild=5-6 [n=8], moderate=7-9 [n=8], severe=10-14 [n=6]), and demographically matched control patients with normal hepatic function (n=14 total). The first patient with severe hepatic impairment was enrolled after ≥2 patients with mild hepatic impairment and ≥2 patients with moderate hepatic impairment had been enrolled and followed for ≥48 hours after dosing to ensure no significant safety signals were observed. On Day 1, patients received a single oral dose of etrasimod 2 mg after ≥10-hour fast. PK and safety data were collected over 21 days. Safety was assessed by adverse event monitoring, vital signs, physical and neurological examination, 12-lead electrocardiogram (ECG), and clinical laboratory evaluations. See FIG. 1. Plasma concentrations of etrasimod were determined via liquid chromatography-tandem mass spectrometry (LC-MS/MS) from matrix-matched calibration curves between 0.25 to 100.0 ng/ml and 0.1 to 120.4 ng/mL for total etrasimod (bound and unbound) and unbound etrasimod, respectively. Analyte LC-MS/MS quantitation reagents included an ACE C18 column, a 20-mM ammonium acetate/acetonitrile mobile phase, a Shimadzu LC pump and autosampler, and a Sciex API6500+MS with an electrospray probe in negative multiple reaction monitoring. Plasma protein-free supernatant was collected via ultracentrifugation at 223,000 g for 4 hours. Unbound etrasimod concentrations were only measured four hours post dose and results assumed to apply to other PK collection times. For protein precipitation, acetonitrile was applied to a 1:1 mixed matrix of plasma: supernatant. The positive control was warfarin. Plasma concentrations and PK parameters were summarized by hepatic function using descriptive statistics. Unbound etrasimod PK parameters were based on total etrasimod PK parameters adjusted by percent unbound. Mean plasma concentration-time profiles were presented graphically. Phoenix WinNonlin (CertaraUSA, Inc.) was used for PK analysis.

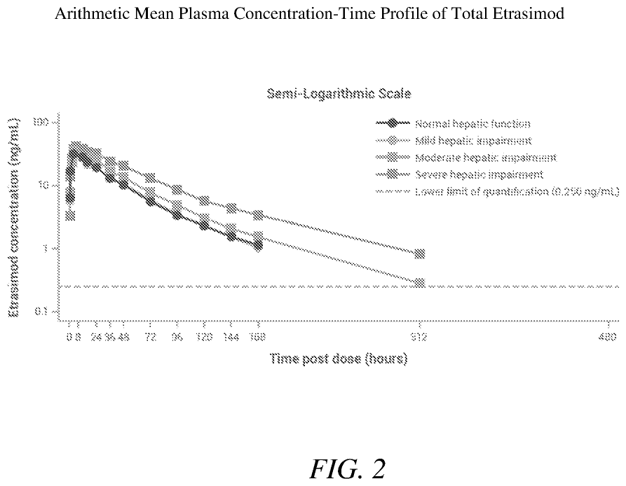

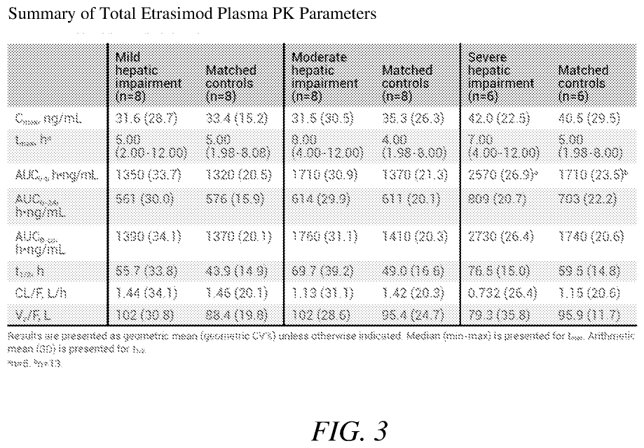

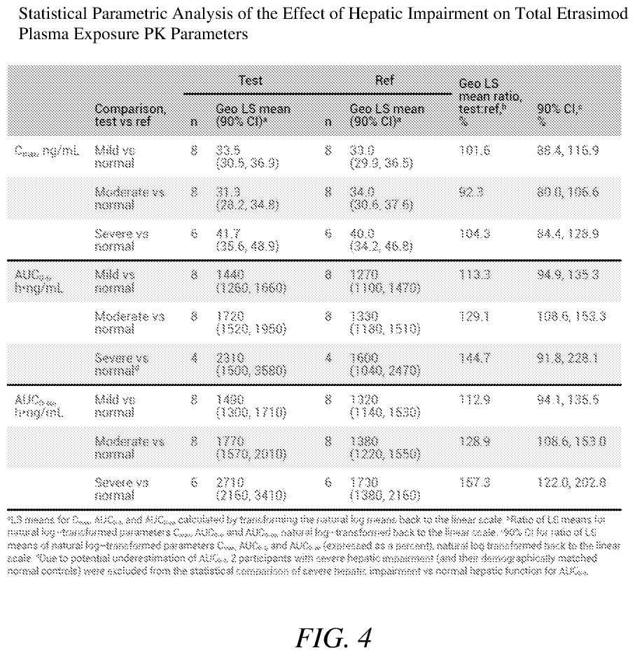

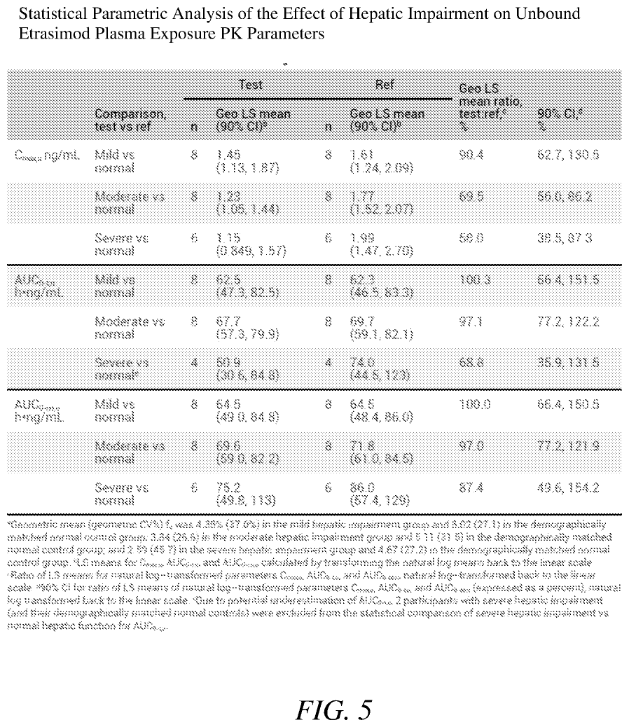

Thirty-six patients were enrolled and dosed; 35 patients completed the study. Etrasimod mean plasma concentration-time profiles overlapped between the normal hepatic function and mild hepatic impairment groups and were slightly higher in the moderate and severe hepatic impairment groups. Across all groups, etrasimod was absorbed with a median tₘₐₓ ranging from 4 to 8 hours. See FIGS. 2-4. When compared with their respective demographically matched normal control groups, single dose etrasimod peak exposure (Cₘₐₓ) was comparable for all hepatic impairment groups, whereas etrasimod total exposure (AUC) measures were progressively higher (from 12.9% to 57.3% higher) in the mild, moderate, and severe hepatic impairment groups. Unbound etrasimod Cₘₐₓ values were progressively lower (up to 42.0% lower) in the mild, moderate, and severe hepatic impairment groups, whereas unbound etrasimod AUC values were typically comparable for all hepatic impairment groups when compared with their respective demographically matched normal control groups. See FIG. 5. Etrasimod t_{1/2} only moderately increased as hepatic function decreased, with mean values ranging from 43.9 to 59.5 hours in the demographically matched normal control groups vs 55.7, 69.7, and 76.5 hours in the mild, moderate, and severe hepatic impairment groups, respectively. A single oral dose of etrasimod was well tolerated. There were no clinically significant safety findings when etrasimod was administered to patients with normal hepatic function or patients with mild, moderate, or severe hepatic impairment.

A single oral dose of etrasimod 2 mg was well tolerated, with relatively modest changes in etrasimod exposure observed in patients with mild, moderate, or severe hepatic impairment. These results suggest etrasimod dose adjustment may not be warranted in patients with hepatic impairment.

1-10. (canceled)

11. A method of treating an S1P1 receptor-associated disorder in a patient having mild, moderate, or severe hepatic impairment, the method comprising: administering to the patient having mild, moderate, or severe hepatic impairment an oral dosage form comprising a dose of Compound 1 or a salt thereof equivalent to 1 mg to 5 mg of Compound 1, wherein the dose is not adjusted for the patient having mild, moderate, or severe hepatic impairment, wherein said Compound 1 is (R)-2-(7-(4-cyclopentyl-3-(trifluoromethyl)benzyloxy)-1,2,3,4-tetrahydrocyclopenta[b]indol-3-yl)acetic acid.

12. The method of claim 11, wherein the patient has mild hepatic impairment.

13. The method of claim 11, wherein the patient has moderate hepatic impairment.

14. The method of claim 11, wherein the patient has severe hepatic impairment.

15. The method of claim 11, where in the dose comprises an amount equivalent to 2 mg of Compound 1.

16. The method of claim 11, where in the dose comprises an amount equivalent to 3 mg of Compound 1.

17. The method of claim 11, wherein the S1P1 receptor-associated disorder is selected from the group consisting of ulcerative colitis, atopic dermatitis, alopecia areatea, eosinophilic esophagitis, and Crohn's disease.
